(19) United States
(12) Patent Application Publication
(10) Pub. No.: US 2026/0009015 A1
(43) Pub. Date: Jan. 8, 2026

(54) Method for Preparing High Purity DNA Fragment Mixture

(71) Applicant: BNC Korea, Inc., Daegu (KR)

(72) Inventors: Wan Gyu CHOI, Gyeonggi-do (KR); Man Sub KIM, Gyeonggi-do (KR); Jae Moon SHIN, Daegu (KR); Kyung Won SEO, Daegu (KR); Eun Kyung LEE, Daegu (KR); Eun Ji LEE, Daegu (KR)

(21) Appl. No.: 19/119,142

(22) PCT Filed: Dec. 19, 2022
(86) PCT No.: PCT/KR2022/020742

§ 371 Date: Apr. 8, 2025

(30) Foreign Application Priority Data
Oct. 12, 2022 (KR) 10-2022-0130968

(51) Int. Cl. C12N 15/10

(52) U.S. Cl. CPC C12N 15/1003

(57) ABSTRACT

The present invention relates to a method for preparing a high purity DNA fragment mixture using isopropanol and magnesium chloride, wherein impurities such as moisture, blood, and fat are effectively removed and the purity of the DNA fragment mixture increases using isopropanol, and a DNA fragment mixture having a relatively narrow molecular weight range of 1,000 KDa to 10,000 KDa can be prepared using magnesium chloride.

### BACKGROUND OF THE INVENTION

### 1. Field of the Invention

The present invention relates to a method of manufacturing a DNA fragment mixture, specifically to a method for preparing a high purity DNA fragment mixture using isopropanol and magnesium chloride.

### 2. Description of the Related Art

A nucleotide is a structural unit that makes up nucleic acids such as DNA and RNA, and a polymer made up of 10 or more nucleotides is called a polynucleotide (PN). In addition, a nucleotide is composed of a combination of one 5-carbon sugar (pentose), one phosphate, and one base (adenine, guanine, thymine, or cytosine), and the balance of combinations that make up nucleotides in trout or salmon is 96.5% identical to that in humans. This high similarity is significant in that it can all be used without any discarded nucleic acids.

Meanwhile, a DNA fragment mixture is a mixture in which DNA exists in the form of fragments with reduced molecular weight. Since the DNA fragment mixture comprises essential components of cells, its value is increasing due to its diverse uses, such as being used for medical devices and pharmaceuticals for the purpose of treating and improving wounds, such as relieving mechanical friction and pain by injecting it into wounds, musculoskeletal areas, or joints, knees, and joint cavities, or as cosmetics, food additives, and biochemical experimental materials for the purpose of improving wrinkles related to cell activity.

As a conventional method for preparing a DNA fragment mixture, Korean Patent Publication No. 10-390529 discloses a method for extracting nucleic acids using an ultrafiltration membrane. However, this method has the disadvantage of being difficult to extract high-purity nucleic acids because it cannot effectively remove impurities such as RNA, protein, fat, and moisture. In addition, Korean Patent Publication No. 10-2019-0065676 discloses a method for reducing DNA molecular weight by adjusting pH, but this method has the disadvantage of being difficult and dangerous because it uses strong acids and strong bases.

Accordingly, the present inventors, while studying a method for preparing a high-purity DNA fragment mixture that safely reduces DNA molecular weight and effectively removes impurities, confirmed that when isopropanol is used to remove impurities and magnesium chloride is used to mix DNA fragments, the DNA molecular weight can be safely reduced with high yield and purity, leading to the completion of the present invention.

### SUMMARY OF THE INVENTION

It is an object of the present invention to provide a method for preparing a high purity DNA fragment mixture.

It is another object of the present invention to provide a high purity DNA fragment mixture.

To achieve the above objects, the present invention provides a method for preparing a high purity DNA fragment mixture comprising the following steps:
(Step 1) a step of treating milt with isopropanol to obtain milt with impurities removed;
(Step 2) a step of treating the above impurity-removed milt by adding it to a sodium chloride aqueous solution;
(Step 3) a step of adding a sodium lauryl sulfate aqueous solution to the sodium chloride aqueous solution containing milt to obtain a primary milt lysate solution in which cells were decomposed and nucleic acids were extracted;
(Step 4) a step of precipitating the primary milt lysate solution with ethanol and separating the precipitate to obtain a secondary milt lysate solution;
(Step 5) a step of adding sodium chloride to the secondary milt lysate solution, and firstly inactivating virus at a high temperature of 80 to 100° C.;
(Step 6) a step of adding magnesium chloride to the virus-inactivated milt lysate solution to obtain a DNA fragment mixture with reduced nucleic acid molecular weight; and
(Step 7) a step of secondarily inactivating virus by treating the DNA fragment mixture with reduced nucleic acid molecular weight with ethanol, and then obtaining a precipitate.

In addition, the present invention provides a high purity DNA fragment mixture prepared by the above method.

### ADVANTAGEOUS EFFECT

The method for preparing a high purity DNA fragment mixture according to the present invention has the effect of effectively removing impurities such as moisture, blood, and fat by using isopropanol to increase the purity of the DNA fragment mixture, and of preparing a DNA fragment mixture having a relatively narrow molecular weight range of 1,000 KDa to 10,000 KDa by using magnesium chloride.

### BRIEF DESCRIPTION OF THE DRAWINGS

FIG. 1 is a photograph showing the comparison of transparency of the DNA fragment mixtures prepared according to the pretreatment methods.

FIG. 2 is a photograph showing the comparison of the molecular weight reduction effect according to the nucleic acid molecular weight reduction method.

FIG. 3 is a photograph showing the comparison of the molecular weight reduction effect according to the magnesium chloride treatment time.

FIG. 4 is a photograph showing the comparison of the molecular weights of Example 1 and the control group (PN, HTL Co.).

FIG. 5A is a graph showing the comparison of the GPC assay results of the control group.

FIG. 5B is a graph showing the comparison of the GPC assay results of Example 3.

FIG. 5C is a graph showing the comparison of the GPC assay results of Example 2.

FIG. 5D is a graph showing the comparison of the GPC assay results of Example 1.

FIG. 6 is a graph showing the comparison of the viscoelasticity of Example 1 and the control group.

### DESCRIPTION OF THE PREFERRED EMBODIMENTS

Hereinafter, the present invention is described in detail.

In one aspect of the present invention, the present invention provides a method for preparing a high purity DNA fragment mixture comprising the following steps:
(Step 1) a step of treating milt with isopropanol to obtain milt with impurities removed;
(Step 2) a step of treating the above impurity-removed milt by adding it to a sodium chloride aqueous solution;
(Step 3) a step of adding a sodium lauryl sulfate aqueous solution to the sodium chloride aqueous solution containing milt to obtain a primary milt lysate solution in which cells were decomposed and nucleic acids were extracted;
(Step 4) a step of precipitating the primary milt lysate solution with ethanol and separating the precipitate to obtain a secondary milt lysate solution;
(Step 5) a step of adding sodium chloride to the secondary milt lysate solution, and firstly inactivating virus at a high temperature of 80 to 100° C.;
(Step 6) a step of adding magnesium chloride to the virus-inactivated milt lysate solution to obtain a DNA fragment mixture with reduced nucleic acid molecular weight; and
(Step 7) a step of secondarily inactivating virus by treating the DNA fragment mixture with reduced nucleic acid molecular weight with ethanol, and then obtaining a precipitate.

In step 1 above, impurity removal is accomplished by adding isopropanol to milt, pulverizing the milt and isopropanol mixture, leaving the mixture for 5 to 30 minutes, and then separating the milt pulverized material and isopropanol.

Said isopropanol can be added in an amount of 3 to 6 times the weight of milt, preferably 4 to 5 times, and more preferably 5 times.

Said milt may be milt of trout or salmon, and is preferably milt of salmon.

Steps 2 and 3 above are the steps in which cell decomposition and nucleic acid extraction are performed. In step 2 above, cells are decomposed at high temperature through a heat treatment method, and nucleic acids are extracted using sodium chloride. In addition, in step 3, sodium lauryl sulfate is used to decompose cells that were not broken down in step 2, as well as to further remove impurities including fat and protein.

More specifically, in step 2 above, an aqueous solution of sodium chloride at a concentration of 20 to 50 wt % is added to the impurity-removed milt, treated at a temperature of 90 to 100° C. for 1 to 4 hours, and then cooled to a temperature of 60 to 65° C. Preferably, an aqueous solution of sodium chloride at a concentration of 25 to 35 wt % is added, followed by treatment at a temperature of 95 to 100° C. for 1 to 2 hours.

In step 3 above, 10 to 20 wt % of a sodium lauryl aqueous solution is added to the sodium sulfate chloride aqueous solution containing milt to make the final concentration of 0.5 to 2 wt %, and the mixture is treated at 10 to 30° C. for 20 to 40 minutes. Preferably, the sodium lauryl sulfate aqueous solution is added to make a final concentration of 1 to 1.5 wt %.

In step 4 above, the primary milt lysate solution is precipitated by adding ethanol to the final concentration of 50 to 80%, and the precipitate is separated to obtain a secondary milt lysate solution.

More specifically, the primary milt lysate solution is filtered through a 50 to 10 μm pore size filter, ethanol is added to the filtrate to a final concentration of 50 to 80%, and the solution is left for 20 minutes to separate the precipitate. Then, the precipitate is dissolved in distilled water to obtain a secondary milt lysate solution.

The first virus inactivation of step 5 is to add sodium chloride to a final concentration of 0.5 to 2 M and treat at a high temperature of 80 to 100° C. for 12 to 20 hours. Preferably, sodium chloride is added to make a final concentration of 0.7 to 1.5 M, and treatment can be performed at a high temperature of 80 to 90° C.

The step 6 is a step of adding magnesium chloride to make the final concentration of 10 to 30 mM and treating at 80 to 100° C. for 2 to 5 hours. Preferably, magnesium chloride is added to make the final concentration is 15 to 35 mM, and treatment can be performed at a high temperature of 80 to 90° C.

In step 6 above, magnesium chloride decomposes nucleic acids and reduces the molecular weight of DNA fragments.

The step 7 is a step of adding 50 to 80% ethanol and treating for 12 to 20 hours.

Specifically, in this step, ethanol is added to a final concentration of 50 to 80%, left for 10 to 30 minutes to separate a precipitate, and then 50 to 80% ethanol is added to the precipitate and treated for 12 to 20 hours. Preferably, 60 to 75% ethanol is added and treated for 15 to 16 hours.

In another aspect of the present invention, the present invention provides a high purity DNA fragment mixture prepared by the above method. The DNA fragment is characterized by having a molecular weight of 1,000 to 10,000 KDa.

In the present invention, “DNA fragment mixture” means PN (polynucleotide) or PDRN (polydeoxyribonucleotide), preferably PN.

In the present invention, “impurities” include blood, moisture, and proteins, fats, endotoxins, and the like that elute due to decomposition of cells of milt.

In the present invention, “treatment” means immersing or stirring.

Hereinafter, the present invention will be described in detail by the following examples and experimental examples.

However, the following examples and experimental examples are only for illustrating the present invention, and the contents of the present invention are not limited thereto.

### EXAMPLE 1

### Preparation of High Purity DNA Fragment Mixture 1

A high purity DNA fragment mixture was prepared according to the following steps.
(Step 1) After thawing 200 g of frozen salmon milt, the blood vessels, blood, eggs, and other internal organs were removed. To the above milt, isopropanol equal to 5 times the weight of the milt was added, ground with a mixer, and allowed to stand for 10 minutes. Then, the milt powder and isopropanol were separated through centrifugation and filtration.
(Step 2) Sterile distilled water was added to the pulverized material of step 1 above and stirred, and then 30% sodium chloride was added and stirred slowly at 95° C. for 1 hour. The reaction mixture was then cooled to 60-65° C.
(Step 3) To the aqueous solution of sodium chloride containing milt, sodium lauryl sulfate was added to a final concentration of 18 and stirred slowly for 30 minutes.
(Step 4) After filtering the lysate solution of step 3 above through a 50 to 10 micrometer pore size filter, ethanol was added to the filtrate to a final concentration of 50 to 80% and left for 20 minutes. After standing, the precipitate obtained was washed by adding 50 to 80% ethanol and slowly stirring. After washing, the precipitate was dehydrated in 95% ethanol and dried using a hot air dryer (55° C.) or a vacuum dryer (45° C., −0.09 to −0.1 MPa). Then, the dried product was completely dissolved in sterilized distilled water to a final concentration of 1 to 3%.
(Step 5) The lysate solution of step 4 above was filtered through a 1 to 0.5 micrometer filter, to which sodium chloride was added to a final concentration of 1 M, followed by stirring slowly at 85° C. for 16 hours.
(Step 6) To the lysate solution of step 5 above, magnesium chloride was added to a final concentration of 20 mM, and stirred slowly at 85° C. for 3 hours and 30 minutes.
(Step 7) The lysate solution of step 6 above was sterile-filtered through a 0.22 micrometer pore size filter. Ethanol was added to the filtrate to a final concentration of 70% and left for 20 minutes to obtain a precipitate. The precipitate was washed by adding 70% ethanol and slowly stirring for 16 hours.
(Step 8) After adding 95% ethanol to the precipitate washed in step 7 above, the mixture was slowly stirred for 10 minutes to dehydrate, and then dried using a hot air dryer (55° C.) or a vacuum dryer (45° C., −0.09 to −0.1 MPa) to prepare a high purity DNA fragment mixture with a molecular weight of 1,000 to 10,000 KDa.

### EXAMPLE 2

### Preparation of High Purity DNA Fragment Mixture 2

A high purity DNA fragment mixture was prepared in the same manner as in Example 1, except that the magnesium chloride treatment in (Step 6) of Example 1 was performed at 85° C. for 2 hours.

### EXAMPLE 3

### Preparation of High Purity DNA Fragment Mixture 3

A high purity DNA fragment mixture was prepared in the same manner as in Example 1, except that the magnesium chloride treatment in (Step 6) of Example 1 was performed at 85° C. for 1 hour.

### COMPARATIVE EXAMPLE 1

A DNA fragment mixture was prepared in the same manner as in Example 1, except that distilled water was added instead of isopropanol in (Step 1) of Example 1.

### COMPARATIVE EXAMPLE 2

A DNA fragment mixture was prepared in the same manner as in Example 1, except that isopropanol was not added in (Step 1) of Example 1.

### COMPARATIVE EXAMPLE 3

A DNA fragment mixture was prepared in the same manner as in Example 1, except that heat treatment was performed at 100° C. instead of adding magnesium chloride in (Step 6) of Example 1.

### COMPARATIVE EXAMPLE 4

A DNA fragment mixture was prepared in the same manner as in Example 1, except that 0.4% SDS was added instead of magnesium chloride in (Step 6) of Example 1.

### COMPARATIVE EXAMPLE 5

A DNA fragment mixture was prepared in the same manner as in Example 1, except that instead of adding magnesium chloride in (Step 6) of Example 1, the pH was adjusted to 4 and the treatment was performed at 85° C. for 20 minutes.

### EXPERIMENTAL EXAMPLE 1

### Comparison of Turbidity and Purity According to Milt Impurity Removal Methods

The turbidity and purity were compared according to milt impurity removal methods of examples in which isopropanol was added, comparative example 1 in which distilled water was added, and comparative example 2 in which nothing was added.

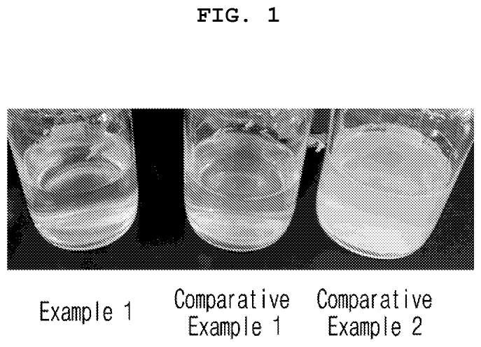

The PN (1 g) prepared according to each milt impurity removal method was hydrated in 100 ml of sterilized distilled water and the turbidity was compared (FIG. 1). As a result, the PN of example prepared by using the method of adding isopropanol was the most transparent. On the other hand, the PN of Comparative Example 1 prepared by adding distilled water and the PN of Comparative Example 2 prepared by not adding anything were opaque white, and in particular, the PN of Comparative Example 2 was the most opaque. Therefore, through the turbidity comparison, it was confirmed that the method for preparing a DNA fragment mixture of the present invention using isopropanol has the best milt impurity removal effect.

TABLE 1

| | | Comparative | Comparative |
|---|---|---|---|
| absorbance | Example 1 | Example 1 | Example 2 |
| 600 nm | 0.0034 | 0.0600 | 0.8300 |
| 280 nm | 0.5704 | 0.6704 | 0.3704 |
| 260 nm | 1.0340 | 1.1340 | 0.6034 |
| 230 nm | 0.4825 | 0.4925 | 0.2925 |
| 260/280 | 1.81 | 1.69 | 1.63 |
| 260/230 | 2.14 | 2.30 | 2.06 |

As shown in Table 1, as a result of comparing the purity of 1 g of the PN prepared according to each impurity removal method after hydrating it in 100 ml of sterile distilled water, it was confirmed that the purity of the PN of Example 1 had the best purity, as the A260/280 value was 1.8 to 1.9 and the A260/230 value was 2.0 to 2.2.

### EXPERIMENTAL EXAMPLE 2

### Comparison of PN Yields According to Milt Impurity Removal Methods

The PN yields were compared according to milt impurity removal methods of examples in which isopropanol was added, comparative example 1 in which distilled water was added, and comparative example 2 in which nothing was added.

TABLE 2

| | Final PN yield |
|---|---|
| Example | 6% (12 g) |
| Comparative Example 1 | 4% (8 g) |
| Comparative Example 2 | 4% (8 g) |

As a result, as shown in Table 2, which shows the final PN yields according to the milt impurity removal methods, it was confirmed that the yield of the PN of example prepared by using the method of adding isopropanol was the highest at 6%.

### EXPERIMENTAL EXAMPLE 3

### Comparison of Molecular Reduction Effects According to Nucleic Acid Molecule Reduction Methods

The molecular reduction effect was compared according to nucleic acid molecular reduction methods of examples in which magnesium chloride was used, comparative example 3 in which heat-treatment was used, comparative example 4 in which SDS was used, and comparative example 5 in which pH was controlled, by electrophoresis analysis of the molecular weight of the PN prepared by each method.

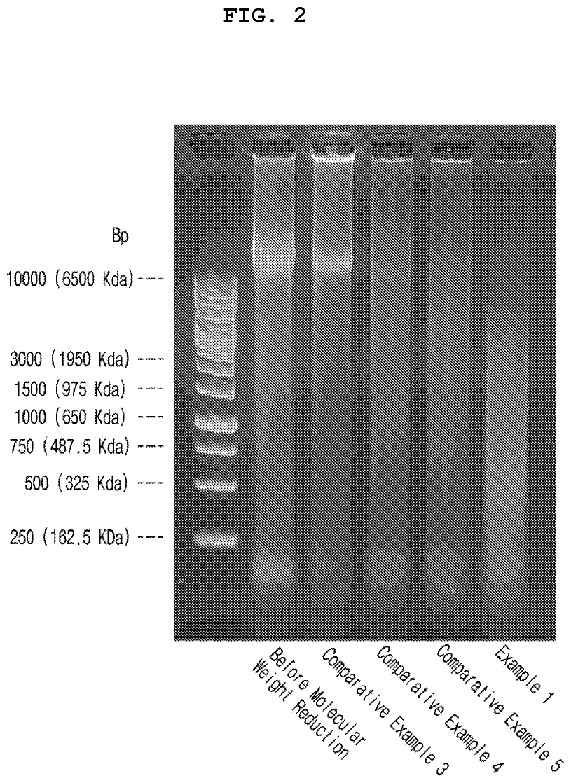

As a result, as shown in FIG. 2, when magnesium chloride was used, the best molecular reduction effect was observed, and in Comparative Examples 3 to 5, no molecular reduction effect was observed or a relatively lower effect was observed compared to the examples. Therefore, it was confirmed that the method for preparing a DNA fragment mixture of the present invention using magnesium chloride has the best molecular reduction effect and can thus be used to produce a DNA fragment mixture having a molecular weight of 1,000 to 10,000 KDa.

### EXPERIMENTAL EXAMPLE 4

### Comparison of Molecular Reduction Effects According to Magnesium Chloride Treatment Time

In Experimental Example 3 above, it was confirmed that the magnesium chloride treatment method had the best DNA molecular weight reduction effect. Therefore, the present inventors further confirmed the DNA molecular weight reduction effect according to magnesium chloride treatment time. For comparison, magnesium chloride was treated for 1 hour (Example 3), 2 hours (Example 2), and 3 hours and 30 minutes (Example 3), respectively, and PN (HTL Co.) was used as a control.

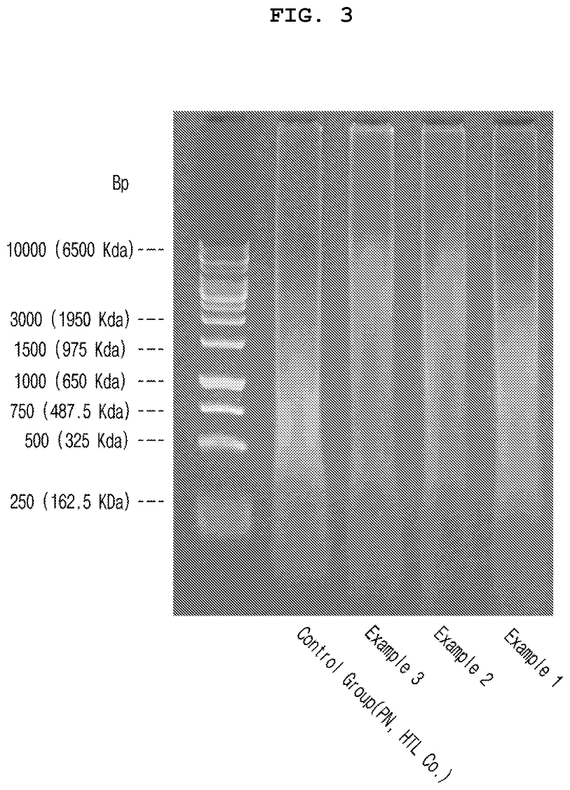

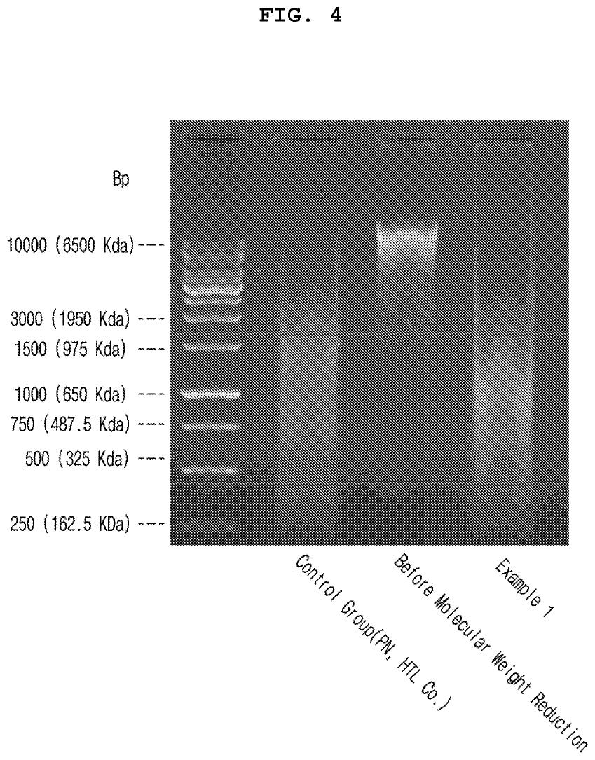

As a result, as shown in FIGS. 3 and 4, the longer the magnesium chloride treatment time, the better the DNA molecular weight reduction effect. In particular, the PN of Example 1 was analyzed to have a DNA molecular weight of 325 to 975 Kda, showing a DNA molecular weight similar to that of the control PN.

### EXPERIMENTAL EXAMPLE 5

### Results of GPC Analysis According to Magnesium Chloride Treatment Time

Following Experimental Example 3, for a more accurate molecular weight comparison, GPC (gel permeation chromatography) was performed according to the magnesium chloride treatment time. Specifically, the analysis was performed using a SHODEX OHpak SB-806M HQ 300 mm column under the following conditions: 40° C. column temperature, 1.0 mL/min rate, RI detector, 1.7 MPa pressure, 100 ul injection. The sample was dissolved in purified water to make 1%, diluted 25 times in mobile phase A, filtered through a 0.45 μm filter, and then analyzed. The standards used at this time were Dextran standard 1 (2,457,000 MW), Dextran standard 2 (1,150,000 MW), and Dextran standard 3 (405,700 MW).

TABLE 3

| | Minimum | Maximum | Average |
|---|---|---|---|
| | molecular weight | molecular weight | molecular weight |
| Control | 947 | 4312 | 2291 |
| Example 3 | 253 | 6563 | 3959 |
| Example 2 | 190 | 4921 | 3116 |
| Example 1 | 766 | 4923 | 2014 |

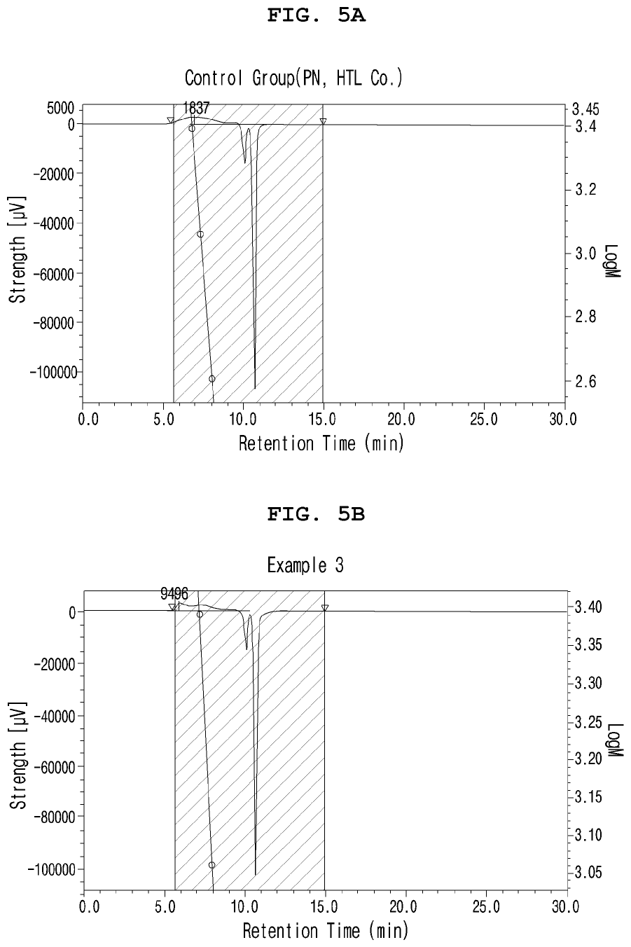

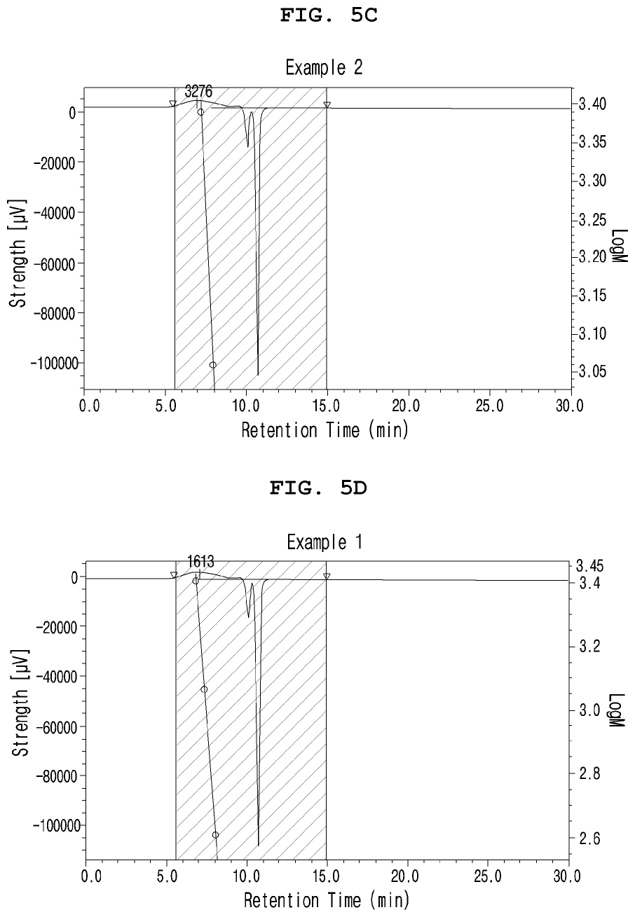

As shown in Table 3 and FIGS. 5A to 5D, it was confirmed that as the magnesium chloride treatment time increased, the range of molecular weights of DNA decreased, allowing the preparation of a DNA fragment mixture of a constant molecular weight, thus inducing PN purification. In addition, compared to the control, it was confirmed that it had a similar average molecular weight.

### EXPERIMENTAL EXAMPLE 6

### Comparison of Viscoelasticity of Example 1 and Control

The viscoelasticity of Example 1 and the control was compared. Specifically, the viscoelasticity of the samples was analyzed using a viscoelasticity apparatus (MCR 92, manufacturer: Anton Paar), a spindle (Measuring Plate PP25 D:25 mm, manufacturer: Anton Paar), a plunger rod, and a spatula. At this time, a reference point was established using Viscosity Standard (Viscosity Standard 1000, Viscosity Standard 5000—manufacturer: BROOKFIELD AMETE), and 1 ml of 1% PN (sample) dissolved in purified water was dispensed into the viscoelasticity apparatus. Then, the storage modulus and loss modulus of each sample were measured at a total of 16 points and analyzed when the angular frequency ω (rad/s) was 1.

TABLE 4

| | Angular Frequency | Storage Modulus | Loss Modulus |
|---|---|---|---|
| | ω (rad/s) | G′ (Pa) | G″ (Pa) |
| Example 1 | 1 | 126.26 | 6.8152 |
| Control (PN, | 1 | 101.48 | 4.0563 |
| HTL Co.) | | | |

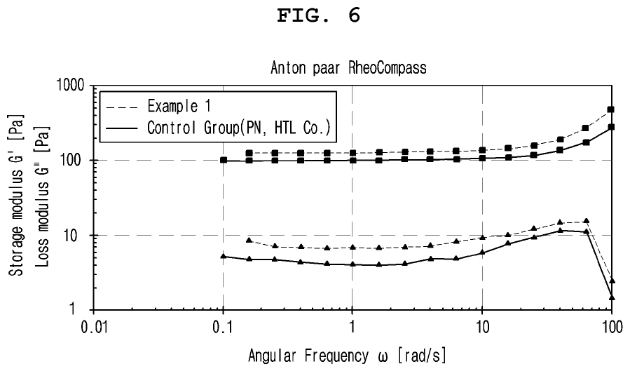

As a result, as shown in Table 4 and FIG. 6, since the storage modulus and loss modulus of Example 1 were higher than those of the control, it was 5 confirmed that the viscoelasticity of Example 1 was superior.

1. A method for preparing a high purity DNA fragment mixture comprising the following steps:
(Step 1) a step of treating milt with isopropanol to obtain milt with impurities removed;
(Step 2) a step of treating the above impurity-removed milt by adding it to a sodium chloride aqueous solution;
(Step 3) a step of adding a sodium lauryl sulfate aqueous solution to the sodium chloride aqueous solution containing milt to obtain a primary milt lysate solution in which cells were decomposed and nucleic acids were extracted;
(Step 4) a step of precipitating the primary milt lysate solution with ethanol and separating the precipitate to obtain a secondary milt lysate solution;
(Step 5) a step of adding sodium chloride to the secondary milt lysate solution, and firstly inactivating virus at a high temperature of 80 to 100° C.;
(Step 6) a step of adding magnesium chloride to the virus-inactivated milt lysate solution to obtain a DNA fragment mixture with reduced nucleic acid molecular weight; and
(Step 7) a step of secondarily inactivating virus by treating the DNA fragment mixture with reduced nucleic acid molecular weight with ethanol, and then obtaining a precipitate.

2. The method for preparing a high purity DNA fragment mixture according to claim 1, wherein the isopropanol of step 1 is 3 to 6 times the weight of the milt.

3. The method for preparing a high purity DNA fragment mixture according to claim 1, wherein the step 2 is adding a sodium chloride aqueous solution at a concentration of 20 to 50 wt % to the impurity-removed milt and treating it at a temperature of 90 to 100° C. for 1 to 4 hours, followed by cooling.

4. The method for preparing a high purity DNA fragment mixture according to claim 1, wherein the step 3 is adding a sodium lauryl sulfate aqueous solution at a concentration of 10 to 20 wt % to a final concentration of 0.5 to 2 wt %, and treating at 10 to 30° C. for 20 to 40 minutes.

5. The method for preparing a high purity DNA fragment mixture according to claim 1, wherein the step 4 is precipitating the primary milt lysate solution by adding ethanol to a final concentration of 50 to 80%, and separating the precipitate to obtain a secondary milt lysate solution.

6. The method for preparing a high purity DNA fragment mixture according to claim 1, wherein the primary virus inactivation in step 5 above is performed by adding sodium chloride to a final concentration of 0.5 to 2 M, and treating at a high temperature of 80 to 100° C. for 12 to 20 hours.

7. The method for preparing a high purity DNA fragment mixture according to claim 1, wherein the step 6 is adding magnesium chloride to a final concentration of 30 to 10 mM, and treating at 80 to 100° C. for 2 to 5 hours.

8. The method for preparing a high purity DNA fragment mixture according to claim 1, wherein the step 7 is adding 50 to 80% ethanol, and treating for 12 to 20 hours.

9. The method for preparing a high purity DNA fragment mixture according to claim 1, wherein the DNA fragment has a molecular weight of 1,000 to 10,000 KDa.

10. The method for preparing a high purity DNA fragment mixture according to claim 1, wherein the milt is salmon milt.

11. (canceled)
